(19) United States
(12) Plant Patent Application Publication
(10) Pub. No.: US 2002/0157159 P1
(43) Pub. Date: Oct. 24, 2002

(54) Gypsophila plant named 'Dangypfirm'

(76) Inventor: Gabriel Danziger, Nir-Zvi (IL)

Correspondence Address: Foley & Lardner, 3000 K Street, N.W., Suite 500, Washington, DC, 20007-5109, US

(21) Appl. No.: 09/838,249

(22) Filed: Apr. 20, 2001

(51) Int. Cl. A01H 5/00

(52) U.S. Cl. PLT/354

(57) ABSTRACT

A new and distinct Gypsophila plant named ‘Dangypfirm’ characterized by having hundreds of semi to full double flowers per stem, flowers arranged in clusters with outstanding stability and open formation, easy handling, prolonged vase life, year round growing season under regular cultural practices, and suitable for both indoor and outdoor cultivation.

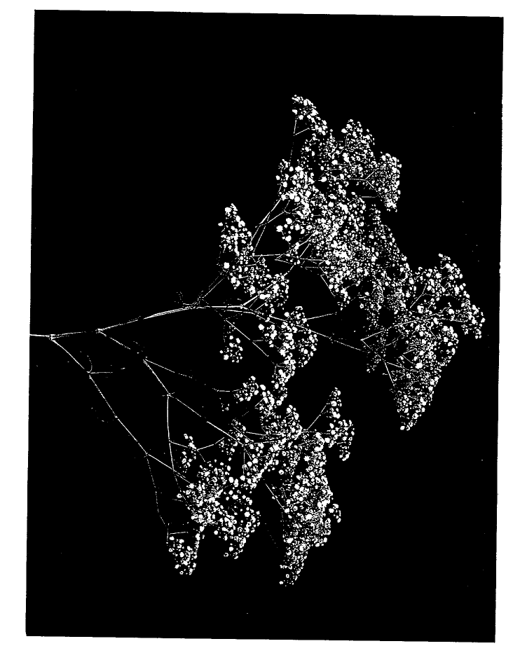

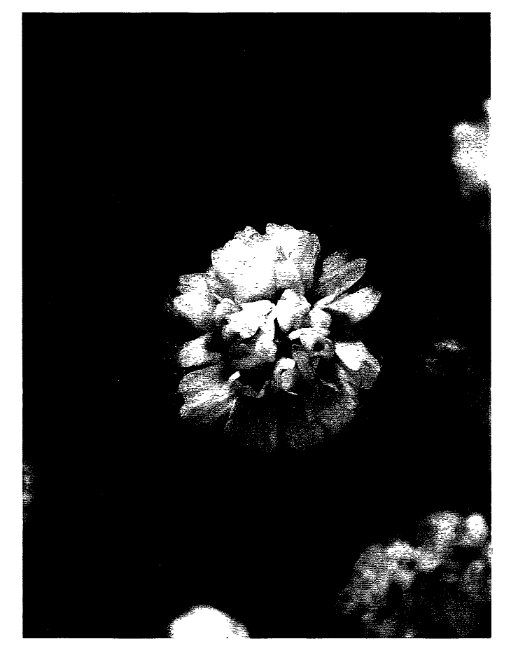

### BACKGROUND OF THE INVENTION

The present invention relates to a new and distinct cultivar of Gypsophila plant, botanically known as Gypsophila paniculata, hereinafter referred to by the cultivar name ‘Dangypfirm’.

The new cultivar was originated from a cross made in a controlled breeding program in Mishmar Hashiva, Israel in 1999. The female parent is Gypsophila paniculata, ‘F. CV. -1087’. The male parent is Gypsophila paniculata, ‘F. CV. -16’. ‘Dangypfirm’ was discovered and selected by the inventor, Gabriel Danziger, as a flowering plant within the progeny of the stated cross in a controlled environment in Mishmar Hashiva, Israel.

Asexual reproduction of the new cultivar was by removing cuttings from the original plant was first performed in a controlled environment in Mishmar Hashiva, Israel in May, 1999, by the inventor and has demonstrated that the combination of characteristics as herein disclosed for the new cultivar are firmly fixed and retained through successive generations of asexual reproduction.

### BRIEF DESCRIPTION OF THE INVENTION

The following traits have been repeatedly observed and are determined to be basic characteristics of ‘Dangypfirm’ which in combination distinguish this Gypsophila as a new and distinct cultivar:

1. hundreds of semi to full double flowers per stem;

2. flowers arranged in clusters with outstanding stability and open formation;

3. easy handling;

4. prolonged vase life; and

5. year round growing season under regular cultural practices, suitable for both indoor and outdoor cultivation.

‘Dangypfirm’ has not been observed under all possible environmental conditions. The phenotype of the new cultivar may vary significantly with variations in environment such as temperature, light intensity, and day length without any change in the genotype of the plant. The following observations, measurements and values describe the new cultivar as grown in Mishmar Hashive, Israel under conditions which closely approximate those generally used in commercial practice.

Of the many commercial cultivars known to the present inventor, the most similar in comparison to ‘Dangypfirm’ is cultivar ‘Tavor’. In comparison to ‘Tavor’, ‘Dangypfirm’ inflorescence formation is in clusters, has a very rigid stem stability, a smaller flower, and higher number of petals.

### BRIEF DESCRIPTION OF THE DRAWING

The accompanying photographic illustrations show the overall appearance of ‘Dangypfirm’ showing the colors as true as is reasonably possible to obtain in colored reproductions of this type.

### DETAILED BOTANICAL DESCRIPTION

The following observations, measurements and values describe the new cultivar as grown in Mishmar Hashiva, Israel under conditions which closely approximate those generally used in commercial practice. Color references are made to The Royal Horticultural Society Colour Chart (RHS), except where general colors of ordinary significance are used.

Classification:

Botanical.—Hybrid of Gypsophila paniculata

Commercial.—Gypsophila ‘New Love’

Propagation: vegetatively by cuttings of side shoots; tissue culture

Plant:

General Appearance and Form.—Height: 90-100 cm Width: 60-70 cm Habit: Upright bush consisting of 6-8 flowering stems forming a rosette with an apical dominance Form: Bush, upright Flowering Response: Long day Flowering Season: Year round when supplementary lighting provided; natural flowering when day length exceeds 12 hours Lastingness of the Individual Bloom: 10-12 days Rooting: Rooting hormone Fragrance: Typical to Gypsophila

Foliage:

Overall Shape of Leaf.—Lanceolate Base: Ovate Tip: Acuminate

Margin.—Entire

Texture.—Smooth

Main Color of Upper Surface.—Mature leaf: Yellow-green RHS 147 A Immature leaf: Yellow-green RHS 147 A

Main Color of Lower Surface.—Mature leaf: Green RHS 137 A Immature leaf: Green RHS 137 A

Venation.—None

Leaf Size.—Length: 9-10 cm Width: 16-20 cm

Leaf Base Shape.—Ovate

Leaf Apex Shape.—Acuminate

Stipules.—None

Inflorescence:

Natural Flowering Season.—During long day conditions

Corolla.—Form: Round Shape: “Cap” shaped Average Number: Hundreds Size: 7-8 mm Petal Number: 55-65 Petal Shape: Spathulated with tip emarginated Petal Markings: None Dorsal Petals: Free Lateral Petals: Free Petal Color:

Upper surface: White RHS 155 C Lower surface: White RHS 155 C

Stem.—Average length: 90-100 cm Average diameter: 4-5 cm Color: Yellow-green RHS 144 A Internode length: 8-9 cm

Spur.—Color: None

Bud.—Response: Long day conditions Color: Green and white Size before opening: 3 mm in diameter Pedicel Length: 10-12 mm Pedicel Color: Green RHS 144 A

Reproductive Organs.—

Stamen.—10

Seeds.—None produced

Fruit.—None produced

Anthers.—Yellow white

Pollen.—Yellow white

Stigma.—2 Color: White

Disease resistance: Unsusceptible to most pests and diseases common in commercial fields, under regular growing conditions

I Claim:

1. A new and distinct Gypsophila plant named ‘Dangypfirm’, substantially as illustrated and described herein. 002.438767
